(19) United States
(12) Patent Application Publication
(10) Pub. No.: US 2021/0299024 A1
(43) Pub. Date: Sep. 30, 2021

(54) Keratin Compositions

(71) Applicant: WOOL RESEARCH ORGANISATION OF NEW ZEALAND INCORPORATED, Christchurch (NZ)

(72) Inventor: Garth Alan Carnaby, Christchurch (NZ)

(21) Appl. No.: 17/264,755

(22) PCT Filed: Jul. 31, 2019
(86) PCT No.: PCT/IB19/56522

§ 371 Date: Jan. 29, 2021

(30) Foreign Application Priority Data
Jul. 31, 2018 (NZ) 744773

(51) Int. Cl. A61K 8/65; A61K 8/02; A61Q 5/02; A61Q 3/02; A61Q 1/10; A61Q 1/06; A61Q 1/12; A61K 8/04

(52) U.S. Cl. CPC A61K 8/65; A61K 8/0241; A61Q 5/02; A61Q 3/02; A61Q 1/10; A61K 2800/412; A61Q 1/12; A61K 8/04; A61K 8/022; A61K 8/0229; A61K 2800/43; A61Q 1/06

(57) ABSTRACT

The invention relates to compositions comprising a keratin pigment material. The keratin pigment material comprises dyed keratin particles. Compositions comprising keratin pigment material such as inks, paints and cosmetic formulations have advantageous properties compared to similar compositions in which conventional pigment materials are used as colourants.

### 1. FIELD OF THE INVENTION

The invention relates generally to compositions comprising dyed keratin particles in which the particles act as a pigment material, colouring the composition. The invention also relates to the use of these keratin pigment materials and compositions them.

### 2. BACKGROUND TO THE INVENTION

The ability to perceive colour is a vitally important part of the human experience. Colour can influence mood, sway thinking, irritate, soothe and give pleasure. Almost as soon as humans started creating products from their natural environment, they sought to alter the colours of these products.

Coloured compounds and compositions (colourants) have been used to impart colour to a wide range of materials such as paints, textiles, cosmetics and foods, across many industries. Colourants are generally characterised as either pigments or dyes. Pigments are particulate materials which are insoluble in the vehicle in which they are mixed. They exist in suspension in a binder material. Dyes, on the other hand, are either liquid or are soluble in their vehicle, existing in solution.

Pigments of natural origin include inorganic compounds, commonly iron oxides (such as ochre, umber and sienna) and organic pigments derived from plant and animal products such as vermilion. Natural pigments have, to some degree, been superseded by synthetic pigments, both inorganic (such as cadmium yellow/orange/red, cobalt blue and phthalocyanine green G) and organic (the azo and diazo compounds).

Colour fastness, compatibility with carrier materials and particle size and shape are important considerations in the application of pigments. Particle size in paint affects the surface finish, with differences in particle size distribution giving rise to flat, eggshell, semi-gloss and gloss finishes. In applications where skin contact is necessary or desirable, such as in many coating, cosmetic and food applications, the particle size of the pigments is necessarily low; often less than 10 microns, and safety and toxicity are of particular importance.

Many pigments, both natural and synthetic, present toxicity challenges. This restricts their use in food and cosmetic applications. Considerations such as colour fastness and particle size can also limit the usefulness of the less toxic options.

Accordingly, there is a need for compositions with the advantageous properties conferred by inclusion of such a pigment material.

It is therefore an object of the present invention to provide compositions comprising such a material, or to at least provide the public with a useful choice.

### 3. SUMMARY OF THE INVENTION

In one aspect the invention provides a composition comprising a keratin pigment material and one or more suitable excipients wherein the pigment material comprises dyed keratin particles.

In another aspect the invention provides a use of dyed keratin particles as a pigment material in a coating composition.

In another aspect the invention provides a method of making a pigmented coating composition comprising combining dyed keratin particles with one or more coating composition excipients.

In another aspect the invention provides a method of making a composition comprising a keratin pigment material and one or more suitable excipients wherein the pigment material comprises dyed keratin particles; the method comprising:
a) chopping keratin-containing animal fibres to form snippets of less than about 5 mm average length;
b) wet milling the chopped snippets to form a slurry;
c) spray drying the slurry produced in step (b) to form a powder;
d) air jet milling the powder produced in step (c);
e) dyeing the powder produced in step (d) to provide a keratin pigment material;
f) adding the keratin pigment material to one or more suitable excipients to produce the composition.

In one aspect the invention provides a composition comprising a keratin pigment material which is obtained by the above method.

In one embodiment, the composition is a coating composition.

In one embodiment, the coating composition is a paint.

In one embodiment the coating composition is an ink, preferably a printing ink.

In one embodiment the coating composition is a cosmetic formulation.

In one aspect the invention provides a method of improving the sensory characteristics of a textile by printing onto the textile, a coating composition comprising a keratin pigment material and one or more suitable excipients wherein the pigment material comprises dyed keratin particles.

In one aspect the invention provides a method of lowering the contact angle of a textile by printing onto the textile, a coating composition comprising a keratin pigment material and one or more suitable excipients wherein the pigment material comprises dyed keratin particles.

In one aspect the invention provides a method of increasing the flame-retardancy of a material by coating the material with a coating composition comprising a keratin pigment material and one or more suitable excipients wherein the pigment material comprises dyed keratin particles.

In one aspect the invention provides a cosmetic formulation comprising a keratin pigment material and one or more dermatologically acceptable excipients, wherein the pigment material comprises dyed keratin particles.

In one aspect the invention provides a method of conditioning skin by applying to the skin a cosmetic formulation comprising a keratin pigment material and one or more dermatologically acceptable excipients, wherein the pigment material comprises dyed keratin particles.

In one aspect the invention provides a method of cleaning and/or styling hair by applying to the hair a dry shampoo comprising a keratin pigment material and one or more dermatologically acceptable excipients, wherein the pigment material comprises dyed keratin particles.

In the above aspects:

In one embodiment the dyed keratin particles have an average diameter of less than 10 microns.

In one embodiment, about 90% of the dyed keratin particles have a diameter of less than 9.8 microns. In one embodiment, about 50% of the dyed keratin particles have a diameter of less than 4.8 microns. In one embodiment, about 10% of the dyed keratin particles have a diameter of less than 1.9 microns.

In one embodiment, about 90% of the dyed keratin particles have a diameter of less than 9.8 microns, about 50% of the dyed keratin particles have a diameter of less than 4.8 microns, and about 10% of the dyed keratin particles have a diameter of less than 1.9 microns.

In one embodiment the dyed keratin particles have a solubility of less than about 5% w/v in water.

In one embodiment the dyed keratin particles have a regain of greater than about 10%, preferably greater than about 12%, more preferably greater than about 15%.

In one embodiment the keratin particles are derived from animal hair/wool/fur. In one embodiment the keratin particles are derived from sheep's wool, goat hair, alpaca hair, cow hair and/or pig bristle. In another embodiment the keratin particles are derived from animal horns, hooves, feathers, and/or scales. In one embodiment the keratin particles are derived from sheep's wool.

### 4. DETAILED DESCRIPTION OF THE INVENTION

### 4.1 Definitions

The following definitions are presented to better define the present invention and as a guide for those of ordinary skill in the art in the practice of the present invention.

The term “about” when used in connection with a referenced numeric indication means the referenced numeric indication plus or minus up to 10% of that referenced numeric indication. For example, “about 100” means from 90 to 110 and “about six” means from 5.4 to 6.6.

The term “comprising” as used herein means “consisting at least in part of”. When interpreting statements in this specification that include that term, the features, prefaced by that term in each statement, all need to be present but other features can also be present. Related terms such as “comprise” and “comprised” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “pigment material” as used herein, means a particulate colourant which imparts colour to a composition in which it is mixed.

The term “dyed keratin particles” as used herein, means particles of keratin protein that have been coloured using a dye, according to standard processes.

The terms “keratin pigment” and “keratin pigment material” as used herein interchangeably refer to a pigment material that comprises dyed keratin particles. In one embodiment, about 90% of the dyed keratin particles present in the keratin pigment have a diameter of less than 9.8 microns. In one embodiment, about 50% of the dyed keratin particles have a diameter of less than 4.8 microns. In one embodiment, about 10% of the dyed keratin particles have a diameter of less than 1.9 microns. In one embodiment, about 90% of the dyed keratin particles have a diameter of less than 9.8 microns, about 50% of the dyed keratin particles have a diameter of less than 4.8 microns, and about 10% of the dyed keratin particles have a diameter of less than 1.9 microns.

The term “flame retardancy” as used herein with reference to a material, means the property by which ignition of the material is prevented and/or further development of ignition is slowed or retarded. The term “conditioning skin” as used herein means improving the health, appearance and/or feel of skin. Skin may be conditioned by increasing its smoothness, softness, moisturisation or other desirable properties.

### 4.2 the Keratin Pigment Material for Use in the Composition of the Invention

Animal fibres, such as wool and other mammalian fibres, are comprised of keratin protein. This protein contributes to many of the properties that make animal fibres desirable for use next to skin and in ingestible products.

Keratin proteins derived from animal fibres such as wool are routinely processed into soluble forms, which are widely used in topical treatments of the hair and skin, and also in dietary supplements. The safety and efficacy of keratin proteins derived from animal fibres such as wool, feathers, horns and hooves, is well established.

Given these properties, the inventors sought to produce a non-toxic pigment material derived from keratin fibres, that would be suitable for use in a range of applications, but particularly where a safe and natural pigment was essential.

Accordingly, in one aspect the invention provides a composition comprising a keratin pigment material and one or more suitable excipients wherein the pigment material comprises dyed keratin particles.

The dyed keratin particles for use in the composition of the invention demonstrate many properties that make them uniquely suited for use as a pigment material for, in particular, coating compositions.

These properties include ease of use, colourfastness and lack of toxicity. Coating compositions including pigment material comprising dyed keratin particles impart favourable properties to the surface, including increasing flame retardancy. When included in cosmetic formulations the keratin pigment materials may improve skin condition.

In one aspect the invention provides a composition comprising a keratin pigment material and one or more suitable excipients wherein the pigment material comprises dyed keratin particles that have an average diameter of less than about 10 microns, a regain of greater than about 10% and a solubility of less than about 5% w/v.

To prepare the compositions of the invention, keratin particles with particular properties are produced from animal fibres using specific preparation techniques. These particles are then dyed and mixed with excipients suitable for the application in which the composition will be used.

### 4.1 Producing Keratin Particles from Animal Fibres

The keratin particles for use in the compositions of the invention are prepared from keratin fibres, which can be obtained from a range of animal sources including animal hair, wool or fur (such as from sheep, goat, alpaca, cow, pig and the like); animal horns, nails, claws and hooves (such as from cattle, goats, antelope); and animal feathers and scales (such as from birds and fish). In one embodiment the keratin particles are derived from sheep's wool.

Animal fibres such as wool are typically greater than 15 microns in diameter, with coarse wool being typically greater than 25 microns in diameter. To convert the fibres into keratin particles for use as pigments in the compositions of the invention, the fibres must first be processed to form a fine powder and then dyed.

Animal fibres can be processed into powder by a range of techniques well known in the art including chopping, milling using dry or wet attritor milling, ball milling, air jet milling, grinding, pan milling, nano collision processes, stone grinding, spray drying, and any combination of these processes.

The person skilled in the art will be able to select the combination of techniques that produces keratin particles with the necessary properties.

A preferred method is described in Rajkhowa, R; Zhou, Q; Tsuzuki, T; Morton, D. A. V; Wang, X; “Ultrafine wool powders and their bulk properties”, Powder Technology; 2012, 224, pp 183-188.

This method uses wet attritor milling, spray drying and air jet milling to prepare ultrafine wool powder.

Basically, the method comprises:
a) chopping keratin-containing animal fibres to form snippets of less than about 5 mm average length;
b) wet milling the chopped snippets to form a slurry;
c) drying the slurry produced in step (b) to form a powder;
d) milling the powder produced in step (c);
   to form keratin particles.

In one embodiment the wet milling is carried out in an Attritor tank. In one embodiment the wool is wet milled for at least 5 hours. Additives such as anti-foaming agents may be added. The wet milling process may be carried out at low or neutral pH. The pH can be adjusted using suitable acids such as, but not limited to, acetic acid. In one embodiment the wet milling is carried out at pH about 3.4 to 3.8, preferably about 3.6.

In one embodiment, the animal fibres are not chemically pre-treated before processing.

The slurry can be dried in step (c) using standard methods in the art including but not limited to, spray drying, freeze drying and air drying. One dried, the powder can be milled using standard methods including but not limited to, air jet milling and milling in a cutting mill.

This method converts coarse animal fibres to fine, spherical, granular particles of less than 10 microns diameter, while maintaining the core insoluble characteristics of the keratin protein. This insolubility enhances the stability of the pigments in compositions based on both aqueous and non-aqueous solvent systems. It also ensures that the pigment does not contribute to a perception of roughness when touched.

Although the use of larger diameter keratin particles is possible, for example with average diameter of 30-50 microns, an average diameter of less than 10 microns is preferred, particularly for use in compositions that will contact human skin, in which the sensorial properties of the composition is important.

As described in Example 1, crossbred New Zealand wool of average fibre diameter 37.5 microns was processed according to the above method, producing particles of average particle size (using a volume-based measurement) of 6.9 microns.

Microscopic examination of the dyed keratin particles produced in Example 1 showed them to be highly porous compared to dense non-porous pigment particles from other sources, such as iron oxides. The keratin particles are also highly porous compared to the source animal fibres and retain a low solubility. The keratin particles produced in Example 1 have a solubility of 1.5% w/v in water. This is only slightly higher than the solubility of the source wool fibre (<1% w/v in water).

In addition, the keratin particles produced in Example 1 retained the moisture vapour interaction properties of the source animal fibre.

The moisture vapour interaction of materials is measured in terms of “regain”. Regain is defined as the weight of water able to be absorbed by a material as a percentage of its dry weight. Wool has a typical regain of 16% under standard conditions of 20° C. and 65% relative humidity.

Regain is one of the properties responsible for the “next to skin” comfort characteristics of wool fibres. Wool regain is much higher than that of synthetic materials such as nylon, which has a typical regain of 4.5%, and an associated undesirable feel.

The keratin particles produced by the above process typically have a regain of greater than 10%.

The essential properties of the keratin particles, such as size distribution, solubility and regain, are not changed when the particles are dyed.

### 4.2 Dyeing Keratin Particles to Produce Keratin Pigments

The keratin particles prepared by the method above can be dyed to a wide range of colours using standard materials and processes for dyeing animal fibres.

For example, dyes suitable for wool dyeing such as acid dyes including Sandolan, Lanasyn, and the Lanasan range. These dyes can be used under typical industrial dyeing conditions to produce keratin particles of a range of colours and shades.

Water-soluble dyes that are suitable for use in making dyed keratin particles include but are not limited to, FDC Red 4, DC Red 6, DC Red 22, DC Red 28, DC Red 30, DC Red 22, FDC Red 40, CD Orange 4, CD Yellow 5, CD Yellow 6, DC Yellow 8, FDC Green 3, DC Green 5, FDC Blue 1, betanine, carmine, copper chlorophyline, methylene blue, anthocyanins, caramel and riboflavin.

In one embodiment, the dye used to make the keratin pigments is FDC Red 40.

The particles prepared in Example 1 were dyed using Acid Green 25, Sandolan Green MF BL along with dye auxiliaries at pH 4.5 at the boil for 45 minutes. This produced strongly dyed particles with excellent dye exhaustion in the liquor during this time. The greater surface area of the keratin particles, relative to the original fibres, and the high degree of porosity led to uptake of the dyes being more rapid than for the source fibres. Dye was also taken up under milder conditions. Using CI Acid Red Dye 88 at 25° C. and pH 4.5, wool typically absorbs very little dye after 2 hours as elevated temperatures are typically required, whereas substantial dye uptake occurred with the keratin particles in less than ten minutes. Dye exhaustion was excellent compared to the source fibres.

Furthermore, the depth of colour achieved in the dyed particles is strong, similar to that achieved by dyeing the source wool materials. This is surprising as smaller particles are typically associated with a greater degree of light back scattering and so paler colours are generally achieved compared to the use of larger materials. The surprisingly strong colour of the keratin particle pigments is believed to be due to the greater surface area of the highly porous particles and the improved dye interaction resulting from the increased sites of dye binding in the particles compared to the source fibres.

Accordingly, the above process can be used to produce dyed keratin particles with properties that confer advantages to coating compositions in which they are included.

In one embodiment, about 90% of the dyed keratin particles have a diameter of less than 9.8 microns. In one embodiment, about 50% of the dyed keratin particles have a diameter of less than 4.8 microns. In one embodiment, about 10% of the dyed keratin particles have a diameter of less than 1.9 microns.

In one embodiment, about 90% of the dyed keratin particles have a diameter of less than 9.8 microns, about 50% of the dyed keratin particles have a diameter of less than 4.8 microns, and about 10% of the dyed keratin particles have a diameter of less than 1.9 microns.

In one embodiment the dyed keratin particles have a solubility of less than about 5% w/v in water.

In one embodiment, the keratin particles for use in the compositions of the invention have a regain greater than 10%, preferably greater than 12%, more preferably, greater than 15%.

### 4.3 Compositions Including Keratin Pigment Materials and their Uses

The dyed keratin particles prepared using the methods outlined herein constitute pigment materials that can be added to a range of coating compositions to impart colour to the composition.

Accordingly, in one aspect the invention provides a composition comprising a keratin pigment material and one or more suitable excipients wherein the pigment material comprises dyed keratin particles.

A wide range of excipients can be included in the composition and are selected for the particular application of the composition.

For example, the compositions of the invention may be inks that can be used in printing applications such as screen printing, ink jet printing and transfer printing. Typical excipients for inclusion into ink compositions include but are not limited to solvents, resins, lubricants, solubilisers, surfactants and fluorescents.

In one embodiment the composition is an ink comprising about 0.1 to about 40% w/v keratin pigment material. In one embodiment the composition is an aqueous-based ink comprising about 0.1 to about 10% w/v, preferably about 5% w/v keratin pigment material. In another embodiment, the composition is a non-aqueous based ink comprising about 0.1 to about 40% w/v keratin pigment material.

In one embodiment the composition is a screen-printing ink. In one embodiment the composition is a gravure ink. In one embodiment the composition is a jet printing ink. In one embodiment the composition is a foil printing ink.

The ink compositions of the invention can be used to print surfaces including paper and textiles, replacing inks that use pigments of synthetic origin or those containing heavy metals such as copper, cobalt or chrome. This improves the environmental profile of the ink and so also of the printed material.

The keratin pigments can be added to any ink formulation including formulations based on aqueous or non-aqueous systems. These include water-based inks, enamels, UV cured inks and Plastisol based inks. The particles may be included at up to 40% (w/v) of an ink formulation, for example in an aqueous polymer-based screen printing formulation where the particles are included at a rate of from about 0.1% to about 10% (w/v) of the ink formulation.

The ink compositions of the invention are particularly suited for printing onto textile surfaces. When printed onto a synthetic textile surface that has a synthetic or unnatural feel, such as a nylon textile, the surface feel of the textile is improved. The ability of the keratin pigment material to interact with moisture vapour in a similar manner to the source fibre imparts a favourable “next to skin” comfort to the textile.

Printing with the ink compositions of the invention also affects the textile's interactions with liquid moisture (see Example 4). By making the surface more hydrophilic, wicking and liquid transfer are improved. A surface's interaction with moisture is characterised by its contact angle, which measures the “wettability” of the surface. The contact angle is the angle where a liquid-vapor interface meets a solid surface. A lower contact angle indicates greater hydrophilicity and therefore better liquid transfer.

Accordingly, the invention provides a method of improving the sensory characteristics of a textile by printing onto the textile a coating composition comprising a keratin pigment material and one or more suitable excipients wherein the pigment material comprises dyed keratin particles.

In one aspect the invention provides a method of lowering the contact angle of a textile by printing onto the textile, a coating composition comprising a keratin pigment material and one or more suitable excipients wherein the pigment material comprises dyed keratin particles.

In one embodiment the method lowers the contact angle of the textile by about 5%.

In one embodiment the coating composition is an ink and the excipients are ink excipients.

In one aspect the invention relates to an ink for textile printing comprising keratin particles that have an average diameter of less than about 10 microns, a regain of greater than about 10% and a solubility of less than about 5% w/v.

In one aspect the invention relates to a method of improving the sensory characteristics of a textile, the method comprising printing onto the textile a printing ink comprising keratin particles that have an average diameter of less than about 10 microns, a regain of greater than about 10% and a solubility of less than about 5% w/v.

In one aspect the invention provides a method of lowering the contact angle of a textile by printing onto the textile, the method comprising printing onto the textile a printing ink comprising keratin particles that have an average diameter of less than about 10 microns, a regain of greater than about 10% and a solubility of less than about 5% w/v.

Inclusion of the keratin pigment to a coating composition of the invention confers other valuable beneficial properties, as well as providing colour.

When included in a coating composition, the keratin pigments comprising dyed keratin particles act as flame retardants.

As observed in Example 9, printing an ink composition of the invention onto a fabric greatly increases its flame-retardancy by reducing the distance travelled by a flame on an ignited textile and increasing the time taken for a flame to travel along a fabric, thereby improving the properties of the fabric.

In one aspect the invention provides a method of increasing the flame-retardancy of a material by coating the material with a coating composition comprising a keratin pigment material and one or more suitable excipients wherein the pigment material comprises dyed keratin particles.

In one embodiment the material is a fabric and the coating composition is an ink which is printed onto the fabric.

In one embodiment the coating composition is a paint which is painted onto the material.

In another embodiment the composition of the invention is a cosmetic formulation.

Pigments typically used in cosmetic formulations are of synthetic or natural origin, and in many cases are metal-based, such as iron oxides, cobalt, chrome or copper salts. Not all pigments are able to be used in all colour cosmetic applications, with restrictions depending on the area of use. For example, use of many pigments around the eye and on the lips is restricted based on skin sensitivity or food safety.

The keratin pigment material provides a similar desirable colour range to existing pigments whilst also providing a skin and food safe alternative, due to its animal fibre source. The keratin pigments are also colourfast. As a result the keratin pigment material can be used as a cosmetic colourant in a broad range of cosmetic applications, such as foundation, blushers, illuminators, face bronzing lotions, creams and powders, loose and pressed powders, mineral powders, eye shadows, eyeliners, eyebrow pencils, kohl and mascara, mineral powders, lipstick, lip glosses, lip pencils, lip plumpers, pots and palettes, nail varnishes and polishes, nail hardeners and strengtheners, nail base and top coat.

The cosmetic formulations of the invention are prepared in accordance with standard practices in the industry, merely replacing the conventional pigment with a keratin pigment material during the formulation process. This can be done in full, to completely replace for conventional pigment, or in part to achieve a blend of conventional pigments and keratin pigment.

The cosmetic formulations of the invention are intended for topical application to the skin and are formulated accordingly, e.g., have suitable viscosity, colour, fragrance and feel properties. The cosmetic formulations may be provided in a variety of forms including but not limited to, emulsions, lotions, milks, liquids, serums, creams, liquids, solids, gels, powders, mousses, ointments, pastes, sticks, sprays, aerosols, foams and pencils.

The keratin pigment material has a low density compared to standard metallic pigments; 0.303 grams per ml or less compared to 5.0 grams per ml or more for iron oxide powders. In personal care formulations this low density leads to greater coverage per unit mass of material than metal-based pigments and a lighter skin feel.

In a similar manner to use in printing applications, the keratin pigment material provides environmental benefits when used in cosmetic formulations as a substitute for synthetic or metal-based pigments.

In one aspect the invention provides a cosmetic formulation comprising a keratin pigment material and one or more dermatologically acceptable excipients, wherein the pigment material comprises dyed keratin particles.

In one aspect the invention relates to a cosmetic formulation comprising keratin particles that have an average diameter of less than about 10 microns, a regain of greater than about 10% and a solubility of less than about 5% w/v.

Dermatologically acceptable excipients are suitable for use in contact with human keratinous tissue without undue toxicity, incompatibility, instability, allergic response or other adverse effects. The dermatologically acceptable excipients may be present in the cosmetic formulations of the invention at an amount of 20% to 99.99% by weight of the formulation. Excipients may be in any suitable form for the type of cosmetic formulation contemplated including but not limited to solutions, dispersions, emulsions and combinations thereof.

The cosmetic formulations of the invention may also include optional ingredients such as, but not limited to, vitamins, minerals, peptides, amines, sugars, oil control agents, flavonoid compounds, anti-oxidants, preservatives, humectants, exfoliating agents, skin lightening agents, sunscreens, film formers, thickeners, pH adjusters, opacifying agents, fragrances, essential oils, lubricants and anti-wrinkle agents.

The cosmetic formulations of the invention have a beneficial effect on skin. Contact with the formulations increases skin conditioning, leaving it feeling smoother and softer. Cosmetic formulations for application to the lips such as lipsticks and lip glosses leave lips feeling moist, unlike many lip products which are known to dry lips.

In one aspect the invention provides a method of conditioning skin by applying to the skin a cosmetic formulation comprising a keratin pigment material and one or more dermatologically acceptable excipients, wherein the pigment material comprises dyed keratin particles.

In one embodiment, skin is conditioned by increasing its smoothness. In one embodiment, skin is conditioned by increasing its softness. In one embodiment, skin is conditioned by increasing its moisturisation.

In one embodiment the cosmetic formulation is a powder formulation, preferably a powder foundation, blusher or eyeshadow. In one embodiment the cosmetic formulation is a lipstick or lip gloss.

Skin conditioning can be assessed by any technique suitable in the art. For example, using a device that measures skin parameters such as moisture, smoothness and trans-epidermal water loss. Other methods include questionnaires to be completed by users of the cosmetic formulation, reporting impact on skin condition in comparison to a suitable control formulation.

In one embodiment, skin is conditioned, with reference to a suitable parameter, by at least 5, 10, 15, 20, 25, 30, 35, 40, 45 or 50% compared to skin to which the cosmetic formulation has not been applied.

In one embodiment the cosmetic formulation of the invention is a cosmetic powder. In one embodiment the cosmetic powder comprises about 2 to about 7 wt % keratin pigment material.

In one embodiment the cosmetic formulation of the invention is a mascara or eyeliner. In one embodiment the mascara or eyeliner comprises about 9 to about 15 wt % keratin pigment material.

In one embodiment the cosmetic formulation of the invention is a lipstick. In one embodiment the lipstick comprises about 9 to about 13 wt % keratin pigment material.

In one embodiment the cosmetic formulation of the invention is a liquid foundation. In one embodiment the liquid foundation comprises about 1 to about 2 wt % keratin pigment material.

In one embodiment the cosmetic formulation is a dry shampoo.

Dry shampoos use fine particles to absorb oil from hair and impart a clean feel and body to the hair. Use of a dry shampoo avoids the wash-and-rinse routine needed for a liquid detergent shampoo which can strip natural oils from the hair. However, regular use of dry shampoo can lead to a build-up of product which is undesirable to the user.

The particles typically used in dry shampoos are starch based and can impart a white colour and/or leave white residue on the hair.

The use of keratin pigment material as described herein instead of starch, provides a dry shampoo that does not suffer from these drawbacks.

Example 8 describes formulation of a dry shampoo comprising a keratin pigment material which brings colour to the formulation while reducing the usual white residue that is generated.

In one aspect the invention provides a method of cleaning and/or styling hair by applying to the hair a dry shampoo comprising a keratin pigment material and one or more dermatologically acceptable excipients, wherein the pigment material comprises dyed keratin particles.

In one embodiment the dry shampoo comprises about 1 wt % keratin pigment material.

Various aspects of the invention will now be illustrated in non-limiting ways by reference to the following examples.

### 5. EXAMPLES

### Example 1: Process of Preparing Pigment Material Comprising Dyed Keratin Particles

Preparing the Keratin Particles 200 g of New Zealand crossbred wool, average fibre diameter of 37.5 microns, was chopped to approximately 10 mm length and loaded into an Attritor tank along with 1500 ml of deionised water. 3 ml of antifoaming agent was added along with dilute acetic acid (to bring the pH level to 3.6). The wool was processed in the attritor for 5½ hrs at 280 RPM. 1000 ml of deionised water was added and the slurry was processed for a further ½ hr. The slurry was dried in a spray dryer, inlet temp—190° C., pump—40%, aspiration 100%. The particle size was measured on a Malvern Mastersizer 2000, d (0.5)=6.904 microns. The final yield of the particles was 134 g.
Colouring with Acid Dye

The following components were mixed into a solution of 1 g/l sodium acetate, set to pH 4.5-5 with acetic acid and combined at a liquor ratio 100:1:
2% on mass of keratin particles of Acid Green 25, Sandolan Green MF BL
1% on mass of keratin particles of Lyogen MF
5% on mass of keratin particles of Glaubers Salt

The mixture was held at 50° C. for 10 minutes then raised 1-2 degrees per minute to the boil. The mixture was held at the boil for 45-60 minutes to allow good exhaustion. Excellent dye exhaustion was observed after 45 minutes.

Colouring with Natural Dye

The following components were mixed into a solution of 2.0 g/l sodium acetate, set to pH 4.5-5 with acetic acid and combined at a liquor ratio 60:1:
2% on mass of keratin particles of Natural Red 4, Carmine
5% on mass of keratin particles of Glaubers Salt
8% on mass of keratin particles of potassium aluminium sulphate as a mordant

The mixture was held at 50° C. for 10 minutes then raised 1-2 degrees per minute to the boil. The mixture was held at the boil for 45-60 minutes to allow good exhaustion. Good dye exhaustion was observed after 60 minutes.

### Example 2: Formulations for Screen Printing

Keratin pigment material prepared in Example 1 was used to create the following screen-printing formulations:

### Pigmented Plastisol Formulation

| Ingredients | Percentage |
|---|---|
| Plasticizer Phthalate free | 30-40% |
| Calcium Carbonate | 15-25% |
| Fume Silica | 1-5% |
| PVC resin | 30-40% |
| Titanium Dioxide | 5-40% |
| Keratin particle pigment | 10-20% |

Pigmented Aqueous Polymer Formulation:

| Ingredients | Percentage |
|---|---|
| Water | 60-85% |
| Acrylic Copolymer | 10-30% |
| Keratin particle pigment | 0.1-10% |
| Auxiliaries | 1-3% |

### Example 3: Screen Printing of a Fabric

10 g of keratin particles prepared according to Example 1 were dyed as follows to make a keratin pigment material:
4% omp Sandolan Blue MF BLN
1% omp Lyogen MF
5% omp Glaubers Salt
1 g/l sodium acetate
pH 4.5-5 with acetic acid
Liquor ratio 60:1
Hold at 50° C. for 10 minutes then raise 1-2 degrees per minute to the boil. Hold at the boil for 45-60 minutes to allow good exhaustion.
Excellent exhaustion at 45 minutes
Rinse at 60:1 liquor ratio at 65° C. for 10 minutes
Dry powder at 65° C.

The 10 g of resulting keratin pigment material was added to 190 g of Permaset Aqua Print Paste (free of colour pigments) to give 5% keratin pigment material in paste. The mixture was stirred by hand then mixed thoroughly with a high shear mixer. The mixture was then applied to a fabric through a 43T screen for textile printing. The printed textile was heat set when dry with an iron at 160° C. for 2-3 minutes.

### Example 4: Contact Angle of Fabric Printed with Composition of the Invention

The screen-printed fabric of Example 3 was evaluated for contact angle performance along with a variation prepared using Permaset Aqua Print Paste containing 10% of the keratin pigment material. For comparison purposes a similar fabric was prepared according to the method of Example 3 in which the Permaset Aqua Print Paste contained 10% of a standard metallic pigment. Samples were placed on a contact angle goniometer and the contact angle was determined by placement of a small drop of distilled water on the surface of the printed fabric. Optical measurement was performed using the contact angle goniometer immediately on placement of the water.

The results demonstrated a contact angle of 134 degrees for the control sample containing 10% standard metallic pigment and 127 degrees for samples containing 5% and 10% of the keratin particle pigment.

### Example 5: Formulations for Gravure Printing

Keratin pigment material prepared in Example 1 was used to create the following gravure ink formulation

| Ingredients | Percentage |
|---|---|
| Keratin particle pigment | 40% |
| Ester soluble vinyl resin | 2.5% |
| Acrylic resin | 17.5% |
| Wax dispersion | 3.0% |
| Stabiliser | 1.0% |
| Ethyl acetate | 10.0% |
| Isopropyl acetate | 16.0% |
| Glycol ether | 10.0% |

### Example 6: Formulation for Foil Printing

Keratin pigment material prepared in Example 1 were used to create the following gravure ink formulation

| Ingredients | Percentage |
|---|---|
| Keratin particle pigment | 25.0% |
| Poly vinyl butyral | 4.0% |
| Dioctyl phthalate | 4.0% |
| Wax dispersion | 3.0% |
| Ethanol | 54% |
| Isopropyl acetate | 10.0% |

### Example 7: Cosmetic Formulations

Keratin pigment material prepared in Example 1 were used to create the following cosmetic formulations.

### Loose Powder Foundation

| Ingredient | Inclusion (%) |
|---|---|
| Talc | To 100 |
| Zinc Stearate | 3.50 |
| Titanium Dioxide | 10.0 |
| Mica | 9.12 |
| Keratin particle pigment | 5.88 |
| Pentaerythrityl Tetraisostearate | 3.50 |
| Isopropyl Mysristate | 1.00 |
| Tocopherol | 0.10 |
| Fragrance | 0.05 |
| Mica, Lauroyl Lysine | 5.00 |

### Pressed Powder Foundation

| Ingredient | Inclusion (%) |
|---|---|
| Talc | To 100 |
| Zinc Stearate | 10.0 |
| Titanium Dioxide | 10.0 |
| Mica | 9.12 |
| Keratin particle pigment | 5.88 |
| Pentaerythrityl Tetraisostearate | 4.00 |
| Isopropyl Mysristate | 1.50 |
| Tocopherol | 0.10 |
| Fragrance | 0.05 |
| Bismuth Oxychloride | 10.00 |

### Pressed Powder Blusher

| Ingredient | Inclusion (%) |
|---|---|
| Talc | To 100 |
| Zinc Stearate | 8.00 |
| Titanium Dioxide | 4.00 |
| Mica | 15.10 |
| Keratin pigment | 4.90 |
| Isopropyl Jojobate, Jojoba | 4.00 |
| Alcohol, Jojoba Esters | |
| Isopropyl Mysristate | 1.50 |
| Tocopherol | 0.10 |
| Mica, Lauroyl Lysine | 5.00 |

### Pressed Powder Eyeshadow

| Ingredient | Inclusion (%) |
|---|---|
| Talc | To 100 |
| Zinc Stearate | 8.00 |
| Titanium Dioxide | 4.00 |
| Mica | 11.50 |
| Keratin pigment | 3.50 |
| Pentaerythrityl Tetraisostearate | 3.00 |
| Isopropyl Mysristate | 1.00 |
| Tocopherol | 0.10 |
| Bismuth Oxychloride | 10.00 |

### Oil/Water Liquid Foundation

| Ingredient | Inclusion (%) |
|---|---|
| Water | To 100 |
| Magnesium Alumnium Silicate | 1.00 |
| Propylene Glycol | 4.00 |
| Xanthan Gum | 0.30 |
| Mineral Oil | 7.00 |
| Dimethicone 200/100cs | 2.00 |
| Sucrose Polysoyate | 2.00 |
| Lecithin | 0.50 |
| Titanium Dioxide | 8.00 |
| Mica | 3.20 |
| Keratin pigment | 1.80 |
| Glyceryl Stearate and PEG-100 Stearate | 4.00 |
| Stearic Acid | 2.00 |
| Liquid Germall Plus | 0.50 |
| Tocopherol | 0.20 |
| Fragrance | 0.05 |

### Water/Silicone Liquid Foundation

| Ingredient | Inclusion (%) |
|---|---|
| C12-15 Alkyl Benzoate | 9.50 |
| Trihydroxystearin | 0.50 |
| Cyclopentasiloxane | To 100 |
| Titanium Dioxide | 7.00 |
| Mica | 1.87 |
| Keratin pigment | 1.13 |
| Cyclopentasiloxane, | 3.00 |
| PEG/PPG-18/18 Dimethicone | |
| Tocopherol | 0.30 |
| Fragrance | 0.02 |
| Water | 25.00 |
| Germaben II | 0.80 |

### Cake Mascara

| Ingredient | Inclusion (%) |
|---|---|
| Stearic Acid | 31.0 |
| Ceresin | 20.0 |
| Carnauba Wax | 12.0 |
| Tribehenin | 5.0 |
| Lanolin Oil | 5.0 |
| Keratin pigment | 10.0 |
| Talc | 5.0 |
| Triethanolamine | 12.0 |
| Preservative | q.s |

### Oil Based Mascara

| Ingredient | Inclusion (%) |
|---|---|
| Isododecane | To 100 |
| Quatemium-18 Hectorite | 4.0 |
| Ethyl Oleate | 4.0 |
| Beeswax (Bleached) | 8.0 |
| Hydrogenated Polycyclopentadiene, Isodecane | 7.5 |
| Carnauba Wax | 1.0 |
| Hydrogenated Polydecene | 20.0 |
| Keratin Particle Pigments | 12.0 |
| Mica | 8.0 |
| Tocopherol | 0.2 |

### Oil/Water Based Mascara (Water Proof Mascara)

| Ingredient | Inclusion (%) |
|---|---|
| Water | To 100 |
| Magnesium Aluminium Silicate | 0.5 |
| Beeswax (Bleached) | 5.0 |
| Carnauba Wax | 1.0 |
| Stearic Acid | 2.0 |
| Cetearyl Alcohol, Ceteareth-20 | 5.0 |
| Isopropyl Myristate | 3.0 |
| Petrolatum | 4.0 |
| Keratin pigment | 12.0 |
| Mica | 8.0 |
| PPG-17/IPDI/DMPA Copolymer | 15.0 |
| Phenonip | 0.9 |
| Triethanolamine | q.s |

### Water/Silicone Mascara

| Ingredient | Inclusion (%) |
|---|---|
| Cyclopentasiloxane, Disteardimonium | 20.0 |
| Hectorie, Propylene Carbonate | |
| Keratin Particle Pigments | 15.0 |
| Cyclomethicone | 20.0 |
| Cyclopentasiloxane, | 10.0 |
| PEG/PPG-18/18 Dimethicone | |
| Hydrogenated Polycyclopentadiene, | 7.5 |
| Isodecane | |
| Polyglyceryl-4 Oleate | 3.0 |
| Water | 15.3 |
| Phenonip | 0.9 |
| Sodium Chloride | 0.7 |
| Cyclomethicone, Trimethylsiloxysilicate | To 100 |

### Liquid Eve Liner

| Ingredient | Inclusion (%) |
|---|---|
| Water | To 100 |
| Hydroxyethylcellulose | 1.0 |
| Sodium Hydroxide (30%) | q.s. |
| Keratin Particle Pigments | 14.0 |
| Styrene/acrylates Copolymer | 30.0 |
| Germall Plus | 0.2 |

### Pencil Eve Liner

| Ingredient | Inclusion (%) |
|---|---|
| Mineral Oil | To 100 |
| Beeswax | 15.0 |
| Candelilla Wax | 8.0 |
| Ozokerite | 5.0 |
| Cetyl Alcohol | 1.5 |
| Keratin pigment | 10.0 |

### Lipstick

| Ingredient | Inclusion (%) |
|---|---|
| Beeswax | 2.50 |
| Carnauba Wax | 1.50 |
| Candelilla Wax | 3.50 |
| Ceresin | 6.50 |
| Isopropyl Palmitate | 5.00 |
| Octyldodecanol | 7.00 |
| Myristyl Lactate | 5.00 |
| Lanolin Oil | 3.75 |
| Hydrogenated Polyisobutene | 10.0 |
| Bis-hydroxyethyloxypropyl dimethicone | 25.0 |
| Castor Oil | To 100 |
| Titanium Dioxide | 6.00 |
| Keratin pigment | 10.05 |

### Lipgloss

| Ingredient | Inclusion (%) |
|---|---|
| Hydrogenated Polyisobutene | To 100 |
| Polyglycerol-6 Dioleate, | 33.0 |
| Hydrogenated Cotton Seed Oil | |
| Butyloctyl Behenate | 22.0 |
| Carnauba Wax | 2.0 |
| Tocopherol Acetate | 0.5 |
| Castor Oil | 9.0 |
| Keratin pigment | 1.7 |
| Bismuth Oxychloride | 0.7 |

### Nail Polish

| Ingredient | Inclusion (%) |
|---|---|
| Toluene | 25.0 |
| Stearalkonium Hectorite | 1.0 |
| n-Butyl Acetate | 30.0 |
| Ethyl Acetate | 10.0 |
| Isopropyl Alcohol | 6.0 |
| Nitrocellulose | 15.0 |
| Toluene Sulfonamide Epoxy Resin | 5.0 |
| Dibutyl Phthalate | 3.5 |
| Camphor | 1.0 |
| Benzophenone-1 | 0.2 |
| Keratin pigment | 0.25 |
| Titanium Dioxide | 0.7 |
| Bismuth Oxychloride | 2.35 |
| Thinner Blend | q.s. |

### Example 8: Dry Shampoo Formulation

Dry shampoos use fine particles to absorb oil from hair and impart a clean feel and body to the hair. The particles used are commonly starch based and can impart a white colour or leave white residue on the hair. Keratin pigment material produced in Example 1 was assessed for oil absorbance and compared to starch particles using jojoba oil as a representative oil. On standing in excess oil until saturation was reached, the keratin pigment absorbed 96 g/100 g of keratin pigment compared to 3 g/100 g of corn starch. The high oil absorbance achieved by the keratin pigment material is a characteristic that makes it a useful ingredient in dry shampoos. Further, keratin pigment materials prepared as described in Example 1 were demonstrated as having a colour more similar to hair, unlike the white starch particles currently used. As a result, the appearance of undesirable white residue was substantially less when keratin pigment material was applied to hair compared to starch. Accordingly, keratin pigment material was added to a standard dry shampoo formulation to bring colour to the formulation and to reduce white residue in use.

### Dry Shampoo Formulation

| Ingredient | Inclusion (%) |
|---|---|
| Butane-Isobutane-Propane | 80 |
| Alcohol denat | 11.7 |
| Oryza Sativa Starch (and) | 5 |
| Cetrimonium Chloride | |
| Quaternium-98 | 1 |
| Keratin pigment | 1 |
| Stearalkonium Bentonite | 0.4 |
| Linoleic Acid, Linolenic Acid, Tocopherol | 0.1 |
| Isopropyl Myristate | 0.4 |
| Parfum | 0.4 |

### Example 9: Flame Retardant Properties of Fabric Printed with Compositions Comprising Dyed Keratin Particles

Unbleached plain weave cotton fabric (Calico) was screen printed with the ink of Example 3, and a comparable Permaset ink using a conventional metal pigment. Four replicates of each of these two fabrics and an untreated control were tested for flame retardancy according to methods described in ISO 6941:2003 Textile fabrics—Burning behaviour—Measurement of flame spread properties of vertically oriented specimens.

The test involves determining time taken for a flame to travel vertically on the test fabric to 3 fixed points (T3 small vertical travel, T2 medium and T1 long vertical travel). If the flame extinguished before the point then ND is recorded.

The results show that the test fabric treated with ink from Example 3 did not burn as much as either the control fabric or the fabric treated with conventional ink. The conclusion was therefore made that the keratin particle pigments produced in Example 3 imparted a flame retardant effect on the fabric.

| Sample | Time to T3 | Time to T2 | Time to T1 |
|---|---|---|---|
| Control 1 | 13.6 s | 22.6 s | ND |
| Control 2 | 13.3 | 22.8 | 33.4 |
| Control 3 | 12.9 | ND | ND |
| Control 4 | 15.2 | 23.6 | ND |
| Conventional pigment 1 | 14.3 | 24.4 | ND |
| Conventional pigment 2 | 14.8 | 24.5 | ND |
| Conventional pigment 3 | ND | ND | ND |
| Conventional pigment 4 | 12.0 | ND | ND |
| Example 3 fabric 1 | ND | ND | ND |
| Example 3 fabric 2 | ND | ND | ND |
| Example 3 fabric 3 | 14.5 | ND | ND |
| Example 3 fabric 4 | ND | ND | ND |

1.-20. (canceled)

21. A method of making a composition comprising a keratin pigment material and one or more suitable excipients wherein the pigment material comprises dyed keratin particles; the method comprising:
a) chopping keratin-containing animal fibres to form snippets of less than about 5 mm average length;
b) wet milling the chopped snippets to form a slurry;
c) drying the slurry produced in step (b) to form a powder;
d) milling the powder produced in step (c);
e) dyeing the powder produced in step (d) to provide a pigment material;
f) adding the pigment material to one or more suitable excipients to produce the composition.

22. The method of claim 21, wherein the composition is a coating composition.

23. The method of claim 22, wherein the coating composition is a paint, an ink, or a cosmetic formulation.

24. The method of claim 21 wherein the dyed keratin particles have an average diameter of less than 10 microns.

25. The method of claim 21 wherein the dyed keratin particles have a solubility in water of less than about 5% w/v.

26. The method of claim 21 wherein the dyed keratin particles have a regain of greater than about 10%

27. The method of claim 21 wherein about 90% of the dyed keratin particles have a diameter of less than 9.8 microns.

28. The method of claim 21 wherein about 50% of the dyed keratin particles have a diameter of less than 4.8 microns.

29. The method of claim 21 wherein about 10% of the keratin particles have a diameter of less than 1.9 microns.

30. The method of claim 21 wherein the keratin particles are derived from sheep's wool.

31. A composition comprising a keratin pigment material which is obtained by the method according to claim 21.

32. A composition comprising a keratin pigment material and one or more suitable excipients, wherein the pigment material comprises dyed keratin particles.

33. The composition of claim 32 wherein the dyed keratin particles have an average diameter of less than 10 microns.

34. The composition of claim 32 wherein the dyed keratin particles have a solubility in water of less than about 5% w/v.

35. The composition of claim 32 wherein the dyed keratin particles have a regain of greater than about 10%.

36. The composition of claim 32 wherein about 90% of the dyed keratin particles have a diameter of less than 9.8 microns.

37. The composition of claim 32 wherein about 50% of the dyed keratin particles have a diameter of less than 4.8 microns.

38. The composition of claim 32 wherein about 10% of the keratin particles have a diameter of less than 1.9 microns.

39. The composition of claim 32 wherein the keratin particles are derived from sheep's wool.

40. A cosmetic formulation comprising a composition of claim 32 wherein the one or more suitable excipients are dermatologically acceptable excipients.
